(19) United States
(12) Patent Application Publication
(10) Pub. No.: US 2026/0234682 A1
(43) Pub. Date: Aug. 13, 2026

(54) METHOD FOR PRODUCING A STABLE FRUCTOOLIGOSACCHARIDE COMPOSITION, FRUCTOOLIGOSACCHARIDE COMPOSITION, AND USE THEREOF

(71) Applicant: BENEO-ORAFTI SA, Oreye (BE)

(72) Inventor: Linda MERTENS, Schaarbeek (BE)

(21) Appl. No.: 19/636,613

(22) Filed: Apr. 1, 2026

Related U.S. Application Data
(62) Division of application No. 18/027,030, filed on Sep. 16, 2021.

(30) Foreign Application Priority Data
Sep. 17, 2020 (EP) 20196590.2

(51) Int. Cl. C12P 19/18

(52) U.S. Cl. CPC C12P 19/18; C12Y 204/01099

(57) ABSTRACT

A stable fructooligosaccharide (FOS) composition is produced from a raw material containing sucrose-by enzymatic activity in aqueous mixture, chromatographically separating non-FOS carbohydrates from the aqueous mixture and evaporating the aqueous mixture to yield a syrupy FOS composition of at least 65° Bx, wherein at least part or essentially all of the organic acids and ions is formed in situ.

### CROSS REFERENCE TO RELATED APPLICATIONS

This application is a divisional application of U.S. patent application Ser. No. 18/027,030, filed Mar. 17, 2023, which in turn is a US National Phase filing of PCT International Application Serial No. PCT/EP2021/075543, filed Sep. 16, 2021, which claims benefit to European Patent Application Serial No. 20196590.2, filed Sep. 17, 2020, the contents of which are incorporated herein by reference.

### TECHNICAL FIELD OF THE INVENTION

The invention relates to a method for producing a stable fructooligosaccharide (FOS) composition and its use. Fructooligosaccharide (FOS) compositions as such are known, and find increasing use in foodstuffs among others. They may conveniently be used for supplying non-sucrose sweetness, as a texturizer, and/or for their prebiotic properties. The invention also relates to a stable fructooligosaccharide (FOS) composition as such.

### BACKGROUND OF THE INVENTION

Fructooligosaccharide (FOS) compounds are in essence inulin compounds having a low degree of polymerization (DP), whereby ‘low’ means a DP ranging from 3 to about 10. It has been known for some time to produce FOS in the shorter end of the chain length spectrum. These so-called ‘short-chain fructooligosaccharides’ or scFOS typically have a DP of 3 to about 5 or 6. Nevertheless, the terms FOS and scFOS are often used interchangeably, also in the context of the present invention.

GB 2072679 for instance discloses an enzymatic method for producing scFOS that uses sucrose (table sugar) as starting material. Sucrose is essentially a disaccharide constituted of glucose (G) and fructose (F) linked together and can thus be written as ‘GF’. The enzymatic process consists of the transfer of F-moieties to the GF and the majority of compounds formed are GFF (having a DP of 3), GFFF (DP4) and GFFFF (DP5). In the process of GB 2072679, free glucose G and fructose F are liberated and removed by converting glucose and fructose to sorbitol and mannitol by catalytic reduction of the scFOS composition.

The known methods of producing a FOS-composition may also include purification steps referred to as demineralisation, refining, polishing, and the like. The known end products are usually in the form of an aqueous solution. It is known that these aqueous product forms have a tendency towards a self-reduction of pH, which is undesirable as it leads to hydrolysis of the FOS compounds.

It is an aim of the present invention to provide a stable scFOS composition. A stable scFOS composition does not show a substantial change, in particular a decrease, in pH upon storage during at least one month, more preferably during at least two months, even more preferably during at least 6 months. A substantial change would be a change, in particular a decrease, of more than 10%, more preferably of more than 5%, even more preferably of more than 4%, even more preferably of more than 3%, even more preferably of more than 2%, and most preferably of more than 1%.

### SUMMARY OF THE INVENTION

The above and other objectives are achieved by providing a method for producing a stable short-chain fructooligosaccharide (scFOS) composition according to claim 1. The method comprises the steps of:
a) providing a raw material containing sucrose;
b) forming an aqueous mixture of the raw material with an enzyme, whereby the enzyme at least has fructosyltransferase activity;
c) exposing the formed aqueous mixture to conditions whereby FOS-forming takes place until the amount of non-FOS carbohydrates in the mixture, such as fructose, glucose and sucrose, constitute at most 50 wt. % of the total amount of carbohydrates in the mixture;
d) optionally deactivating the enzyme;
e) chromatographically separating non-FOS carbohydrates from the aqueous mixture using a resin, preferably a cationic resin, to yield a FOS-enriched stream containing at least 75 wt. % scFOS relative to the total amount of carbohydrates in the aqueous mixture and a stream enriched in non-FOS carbohydrates, and whereby the FOS-enriched stream comprises at least 100 mg/kg ° Bx of organic acids and ions; and
f) evaporating the FOS-enriched stream after step e) to yield a syrupy FOS composition of at least 65° Bx,
wherein at least part or essentially all of the organic acids and ions is formed in situ during any one of the steps c) to f).

An advantage of the method according to the invention is that it does not require the addition of stabilizers in extra separate steps of the method.

Fructooligosaccharide (FOS) belongs to the compounds known as inulin. Inulin is a generic term that relates to a carbohydrate material consisting mainly of fructose moieties linked via β (2→1) type fructosyl-fructose links, with optionally a glucose starting moiety. Inulin is usually polydisperse, i.e. a mixture of compounds of various chain lengths whereby the degree of polymerisation (DP) of the individual compounds can range from 2 to 100 or higher. The term fructooligosaccharide—abbreviated as FOS—thus indicates a specific form of an inulin material, either monodisperse or polydisperse, whereby the DP of the individual compounds ranges from 2 to 10, in practice often from 2 to 9, or from 2 to 8 or from 2 to 7. Commercially available scFOS is usually a polydisperse material having a number-averaged degree of polymerisation (DP) of about 2 to 4. In practice, FOS is also referred to as oligofructose. As used herein, the terms fructooligosaccharide and oligofructose are considered to be synonyms.

According to the invention, a stable FOS composition is formed. As understood herein, a FOS composition means a composition that contains FOS—either monodisperse or polydisperse—as biggest dry matter constituent and that furthermore may contain other compounds. Examples of such other compounds are: water, sucrose, fructose, glucose, inulin compounds other than FOS, maltose, organic salts, and inorganic salts. It is noted, however, that as used in the context of the present invention and when the FOS composition is intended for consumption by a human or an animal, the term FOS composition is to be regarded as an ingredient for a foodstuff rather than as a foodstuff itself. The creation of a foodstuff is thus a further step whereby the FOS composition serves as a food ingredient.

The method according to the invention is readily executed on an industrial scale. As used herein, the term industrial scale means that the method may be carried out in an installation that is able to process at least 500 kg of raw materials per 24 hours of operation, and preferably at least 1,000 kg per 24 hours up to 1,000,000 kg per 24 hours, and even more. Transferring laboratory findings into large-scale practice may entail many problems, such as scaling, turbidity, foaming, contamination with microbiological pathogens, and the need for purification steps. All these problems may seriously affect the stability of the produced FOS composition. The invented method yields a stable FOS composition on any scale, including an industrial scale.

In step a) of the method according to the invention, a raw material is provided. This raw material should contain, or preferably consists essentially of, sucrose. The sucrose may be provided in (refined) beet sugar, or may be provided in (refined) cane sugar for instance.

The term ‘consist(ing) essentially of’ and equivalents have the usual meaning in the context of a composition in that in addition to compounds that are mandatory other compounds may also be present, provided that the essential characteristics of the composition are not materially affected by their presence.

In step b) of the method according to the invention, an aqueous mixture is formed consisting essentially of the raw material, water and an enzyme. The enzyme is provided to catalyse the formation of FOS from sucrose, which aim may be achieved by selecting an enzyme having at least fructosyltransferase activity. Such enzymes are known per se, for instance as categorised under enzyme category number EC 2.4.1.99 or EC 2.4.1.9. Furthermore, it is known that some β-fructofuranosidases, i.e. enzymes categorised under EC 3.2.1.26, can also have fructosyltransferase activity. These may thus also be suitable in the method according to the invention.

Other suitable enzymes may be added to the mixture, such as those having an endo-inulinase activity. Such enzymes, classified under EC 3.2.1.7, may in the presence of sucrose and an enzyme having at least fructosyltransferase activity give rise to the formation of FOS.

Suitable enzymes for use in step b) of the invention comprise Novozyme 960, supplied by Novozymes, and Pectinex Ultra SP-L, also supplied by Novozymes. A combination of two or more enzymes of which at least one has fructosyltransferase activity may be used. The enzyme may partly or completely be used in immobilized form; it may then be reused several times. It is also possible however to provide the enzyme partly or completely in non-immobilized form.

The amount of enzyme needed in the method according to the invention may be selected by one skilled in the art on the basis of various factors such as the temperature in steps b) and c) of the method, the amount of raw materials, pH, time, and desired conversion rates. These and other relevant factors may be determined by the person skilled in the art following the generally accepted procedures in this technical field.

When forming the mixture, it is preferred that water is present or added so that water becomes the continuous phase in the mixture. If desired, water may also be added in subsequent step c).

In step c) of the invention, the aqueous mixture of step b) is exposed to conditions whereby FOS-forming takes place for an amount of time such that a desired amount of FOS has been formed. In embodiments, it may be helpful to submit the mixture to a heat treatment at a temperature between 60°-100° C., in order to prevent pathogens from being contained or remaining in the mixture. According to the invention, FOS-forming takes place until the amount of non-FOS carbohydrates in the mixture, such as fructose, glucose and sucrose, constitute at most 50 wt. % of the total amount of carbohydrates in the mixture, more preferably at most 45, 40, or 35 wt. % of the total amount of carbohydrates in the mixture. Due to the circumstance that a certain amount of free glucose will typically be formed in the method, a limit may exist on the maximum amount of FOS obtainable in the mixture. Thus, the amount of FOS in the mixture at the end of step c) of the method is preferably at most 65 wt. % of the total amount of carbohydrates.

Conditions to which the aqueous mixture may be subjected such that FOS-forming is to take place in step c) of the method are known as such and one skilled in the art will not experience any difficulties in setting the right conditions. Such conditions, in an embodiment of the method, preferably include a temperature between 40° C.-75° C. and a solids content lying between 40° Bx and 70° Bx, more preferably between 45 and 70° Bx, and more preferably between 5° and 70° Bx.

The wording ‘between’ end-points of a range is construed as encompassing the end points of the range.

In a more preferred embodiment, the temperature ranges from 50° C.-65° C. and the solids content lies between 50° Bx and 65° Bx.

As is known, the unit ‘° Bx’ denotes ‘degrees Brix’ and is widely used in the sugar industry to indicate the solids content of a sugar solution, as derived from its refraction index. As used herein, the same method is used on samples of sucrose and of FOS compositions. The numerical ° Bx values are generally very close to, or even essentially identical to, weight percentages; thus in an alternative expression of the present invention all ° Bx values mentioned in the present description and claims may be read as weight percentages of dry matter.

It has been found that the temperature of the aqueous mixture in step c) when lower than 56° C. may favour microbiological contamination risks for some mixture compositions. By contrast, increasing the solids content of the mixture to above 60° Bx or 65° Bx may favour conditions for inhibiting the growth of undesirable microorganisms for some mixture compositions.

The enzyme or combination of enzymes used in the method of the invention may be provided partly or completely in immobilized form. The method may also be operated in a particularly efficient manner if in step c) the enzyme is not immobilized. A person skilled in the art will know how to provide an enzyme in immobilized or mobilized form.

The enzymatic action in step c) of the method will lead to the formation of FOS, and typically leads to the formation of non-FOS carbohydrates such as in particular free glucose. In a preferred embodiment of the method, the pH during the execution of step c) is controlled within a pre-determined range. The precise range of pH is, as the skilled person knows, dependent on several factors such as in particular the choice and characteristics of the enzyme as used. Control of the pH may be executed by means that are known to the skilled person as such.

The duration of step c) may generally be chosen in function of the amount of FOS that is desired. A suitable duration for this purpose is often selected between 1 and 72 hours, more preferably between 5 and 50 hours, even more preferably between 12 and 36 hours.

Upon completion of step c) of the invention, it may be desirable to ensure that the enzyme is deactivated. An enzyme deactivating step d) may thus optionally be implemented. Deactivation of the enzyme may particularly be preferred when the used enzyme is not immobilized, or partly so. The deactivation of the enzyme may be achieved by methods that are known per se, and may differ for each specific type of enzyme. An exemplary deactivation comprises increasing the temperature to a level of 80, 85 or 90° C., followed by a residence time of typically between 5 and 30 minutes at said increased temperature. Bacterial presence may also be reduced substantially at such a temperature. In another method, the pH of the reaction mixture is increased to above 8 for instance to end enzymatic activity.

After completion of step c), and optionally of step d), a portion of the non-FOS carbohydrates are in step e) chromatographically separated from the aqueous mixture using a resin, preferably a cationic resin, to yield a FOS-enriched stream containing at least 75 wt. %, preferably at least 85, 90, or even 95 wt. % FOS relative to the total amount of carbohydrates, and whereby the FOS-enriched stream comprises at least 100 mg/kg ° Bx of organic acids and ions, wherein at least part or essentially all of the organic acids and ions is formed in situ during any one of the steps c) to f).

The amount of organic acids and ions is defined in mg/kg ° Bx, which means an amount in mg per kg dry matter.

It has turned out that the presence of a mixture of organic acids and ions in the claimed amounts can yield a stable syrupy FOS composition.

The mixture of organic acids and ions may be provided in the FOS composition through different sources. In a preferred method, the organic acids and ions are formed in situ during step e). It has been found that a particularly stable FOS composition may be obtained in this way, and it is believed that the chromatography resin, preferably a cationic resin, may provide or facilitate the formation of at least part of the organic acids and ions, more preferably at least half of the organic acids and ions, and most preferably substantially all of the organic acids and ions.

In another embodiment of the invention, a method is provided wherein in step e) the FOS-enriched stream is brought to a pH within a range of 6.0 to 11.0, preferably 8.0 to 10.0, to form the FOS-enriched stream comprising at least 100 mg/kg ° Bx of organic acids and ions.

In yet another embodiment, a portion of the stream enriched in non-FOS carbohydrates is brought to a pH within a range of 6.0 to 11.0, preferably 8.0 to 10.0, and subsequently recombined with the FOS-enriched stream to form the FOS-enriched stream comprising at least 100 mg/kg ° Bx of organic acids and ions. The said portion of the stream enriched in non-FOS carbohydrates can for example be between 5 and 50% of the stream enriched in non-FOS carbohydrates.

The duration of the pH change may be selected within a broad range. An embodiment of the method wherein the pH is held within the range for a residence time of between ten minutes and eight hours is preferred, more preferably between 30 minutes and eight hours and even more preferably between one hour and six hours.

Recombining the stream enriched in non-FOS carbohydrates with the FOS-enriched stream may alter the pH of the FOS-enriched stream to some extent, depending on the relative amounts of the non-FOS carbohydrates and the FOS. A further improved method according to an embodiment comprises bringing the pH of the FOS-enriched stream and/or of the syrupy FOS composition to below 8.0, more preferably within a range of 6.0 to 8.0, after step e) or step f). This may minimize undesirable side effects such as for example an undesirable increase in ICUMSA color.

The desired stability has been observed when the amount of the organic acids and ions in the FOS-enriched stream is at least 100 mg/kg ° Bx. The stability may further be enhanced in embodiments of the method wherein the amount of the organic acids and ions in the FOS-enriched stream comprises at least 200 mg/kg ° Bx, more preferably at least 300 mg/kg ° Bx, even more preferably at least 400 mg/kg ° Bx, even more preferably at least 500 mg/kg ° Bx, even more preferably at least 1000 mg/kg ° Bx.

The desirable stability is also observed in embodiments of the method wherein the syrupy FOS composition comprises between 1-10 g/kg ° Bx, more preferably between 1.2-8 g/kg ° Bx, and most preferably between 1.5-5 g/kg ° Bx of organic acids and ions. These embodiments may require adding a part of the organic acids and ions to the aqueous mixture during or after step e), during step f) or after step f).

Step f) of the method involves evaporating the aqueous mixture to yield a syrupy FOS composition of at least 65° Bx. Evaporating water from the aqueous mixture may conveniently be carried out by heating, for example to a temperature chosen somewhere between 85° C. and the boiling point of the FOS composition, or even higher. Evaporation should preferably be carried out as long as to obtain a syrupy FOS composition of at least 65° Bx and at most 80° Bx.

An even more improved stability may be obtained in embodiments of the method in which the step f) of evaporating the aqueous mixture is carried out to yield a syrupy FOS composition of at least 67° Bx, more preferably of at least 70° Bx, and most preferably of at least 72° Bx.

Some steps of the method according to the invention may involve adding anti-scaling agents, anti-foaming agents, bactericides, biocides, and flocculants, if desired.

When executing a method for the preparation of a FOS composition, one skilled in the art has at his disposal a number of parameters that may be varied in order to optimise the method and the resulting FOS composition. Among these parameters rank the temperature at which the synthesis reaction is carried out, the solids content of the mixture in steps b) and c), the duration of the enzymatic reaction, and the amount of enzyme added. The method may produce a FOS composition having a relatively high amount of FOS with a degree of polymerisation (DP) of 3, relative to the total amount of fructooligosaccharides; and/or a relatively low amount of FOS having a DP of 7 or higher, relative to the total amount of fructooligosaccharides. Preferably, at most 3 wt. %, more preferably at most 2, 1, or even 0.50, 0.40, 0.30 or 0.20 wt. % of the carbohydrates in the FOS composition consists of oligosaccharides having a DP of 7 or more.

The specific mixture of organic acids and ions present in the FOS surprisingly yields a stable FOS composition after step e) of the method, and particularly after step f) of the method.

In a preferred embodiment of the method, the organic acids comprise at least two of pyrrolidone carboxylic acid (PCA), lactate, acetate, formiate, and citrate. More preferably the organic acids comprise at least two, even more preferably at least three or even all of pyrrolidone carboxylic acid (PCA), lactate, acetate, and formiate. Even more preferably the organic acids comprise at least three of pyrrolidone carboxylic acid (PCA), lactate, acetate, formiate, and citrate, or even at least four of pyrrolidone carboxylic acid (PCA), lactate, acetate, formiate, and citrate, and most preferably pyrrolidone carboxylic acid (PCA), lactate, acetate, formiate, and citrate.

It is preferred that the sum of pyrrolidone carboxylic acid (PCA), lactate, acetate, formiate, and citrate accounts for at least 25 wt. % of all organic acids in the FOS composition or syrupy scFOS composition. More preferably the sum of pyrrolidone carboxylic acid (PCA), lactate, acetate, formiate, and citrate accounts for at least 40, 50, 60, 70, or even at least 80 or 90 wt. % of all organic acids in the FOS composition or syrupy scFOS composition.

A method according to an embodiment of the invention wherein at least acetate, preferably at least lactate and acetate, are not added to the aqueous mixture in a separate method step is preferred. They may however be present in the mixture through another source, such as for example by means of the in situ method as described above.

In other embodiments, the ions and/or the organic acids are not added to the aqueous mixture in a separate method step.

It was further found that an embodiment of the method wherein the ions comprise cations comprising at least one of sodium and potassium, and anions comprising at least one of chloride, nitrate and sulphate, can be instrumental in obtaining good stability of the FOS composition, in particular in combination with the organic acids disclosed above.

In a preferred embodiment of the method, the ions comprise cations that comprise sodium and/or potassium. In another preferred embodiment of the method, the anions comprise at least two of chloride, nitrate and sulphate, and, even more preferred, comprise chloride, nitrate and sulphate.

The resin in step e) is preferably a cationic resin and may then be chosen within a wide range of available cationic resins. Cationic resins belong to the group of ion-exchange resins, i.e. organic compounds that have been synthetically polymerized and contain positively or negatively charged sites that can attract an ion of opposite charge from a surrounding solution. Polymers containing acid groups are classified as acid, or cation, exchanging resins because they exchange positively charged ions, such as hydrogen and metal ions; those containing ammonium groups are considered basic, or anion, exchanging resins because they exchange negatively charged ions, usually hydroxide ions or halide ions. Suitable cationic resins include but are not limited to methacrylic acid divinylbenzene and styrene divinylbenzene copolymers, and phenol-formaldehyde polymers for instance. An embodiment of the method wherein the cationic resin comprises a styrene divinylbenzene copolymer is particularly preferred. The electrically charged groups in the cationic resin may comprise sulfonic and/or carboxylic acid salts.

The FOS composition produced by the method in accordance with the invention may contain compounds other than FOS that cause a characteristic color and flavor. Furthermore, the method for producing the FOS composition may itself introduce compounds other than FOS into the FOS composition that causes the coloring.

As a result of the above referred factors, it was found that an embodiment of the method wherein the fructooligosaccharide (FOS) composition has a color of between 1,000-3,000 Icumsa units can yield a stable FOS composition.

In another embodiment of the method, a FOS composition is produced having a conductivity of between 200-2,000 μS/cm.

Icumsa units as indicator for color are widely used in the sucrose-producing industry, and the Icumsa color may conveniently be measured by method GS2/3-9 (2005), which is in essence a measurement of the absorption of an aqueous solution of sucrose at a wavelength of 420 nm. The measured absorption is re-calculated into Icumsa values. Low Icumsa values indicate a colorless/white color, while higher Icumsa values are indicative of a product having a color in the yellow to brownish range. The method GS2/3-9 (2005) is also used herein for the determination of the color of the produced FOS composition, with the following comments/modifications:
The pH is regulated to 6 (not to 7 as in the standard);
The formula for determining the Icumsa color of a sample is:

$$Icumsa color = ( 100000 \times A ) / ( ° Bx \times ρ \times b )$$

wherein A is the absorption at 420 nm, ° Bx is the solids content in degrees Brix, ρ is the density of the sample, b is the length of the absorption path, and x denotes ‘multiplied by’.

The conductivity of the FOS composition is determined on an aqueous FOS composition having a solids content of 28° Bx, and expressed in microSiemens per centimeter (μS/cm).

A further improved method according to an embodiment further includes a step g) after step e) and before step f) of treating the aqueous mixture with active carbon to remove at least part of colored components present in the aqueous mixture without substantially affecting the minimum amount of 100 mg/kg ° Bx of organic acids and ions in the FOS.

Such an embodiment may influence the color of the FOS composition, wherein, in preferred embodiments, treating the aqueous mixture with active carbon yields a color of between 50-750 Icumsa units, more preferably of between 100-500 Icumsa units, and most preferably of between 150-400 Icumsa units.

Although the invented method may involve other steps than elucidated above, a preferred embodiment of the method does not include any further steps after step f) or step g).

Another aspect of the invention relates to a Fructooligosaccharide (FOS) composition as such. The FOS composition comprises at least 75 wt. % FOS relative to the total amount of carbohydrates, preferably at least 80, 85, 90, or even 95 wt. % FOS relative to the total amount of carbohydrates; the FOS composition comprises at least 100 mg/kg ° Bx of organic acids and ions. In more preferred embodiments, the FOS composition comprises at least 200 mg/kg ° Bx of the organic acids and ions, more preferably at least 300 mg/kg ° Bx, even more preferably at least 400 mg/kg ° Bx, even more preferably at least 500 mg/kg ° Bx, even more preferably at least 1000 mg/kg ° Bx of the organic acids and ions. In other embodiments, the FOS composition comprises between 1-10 g/kg ° Bx, more preferably between 1.2-8 g/kg ° Bx, and most preferably between 1.5-5 g/kg ° Bx of the organic acids and ions. The FOS composition is obtainable by a method in accordance with the invention, as exemplified by the above described embodiments. The FOS composition may be in solid form, or in the form of a—preferably aqueous—slurry, dispersion or solution. The FOS composition can be a scFOS composition.

In a useful embodiment of the FOS composition according to the invention, the organic acids comprise at least one of pyrrolidone carboxylic acid (PCA), acetate, and formiate. In a preferred embodiment of the FOS composition, the organic acids comprise at least two, more preferably at least three, most preferably all of pyrrolidone carboxylic acid (PCA), acetate, and formiate. In a further preferred embodiment of the FOS composition, the organic acids comprise at least two, even more preferably at least three of pyrrolidone carboxylic acid (PCA), acetate, and formiate. In a preferred embodiment, however, the FOS composition does not comprise at least one of citrate, lactate and acetate.

It is preferred that the sum of pyrrolidone carboxylic acid (PCA), optionally lactate, acetate, formiate, and optionally citrate accounts for at least 25 wt. % of all organic acids in the FOS composition. More preferably the sum of pyrrolidone carboxylic acid (PCA), optionally lactate, acetate, formiate, and optionally citrate accounts for at least 40, 50, 60, 70, or even at least 80 or 90 wt. % of all organic acids in the FOS composition.

In another useful embodiment, the ions in the FOS composition comprise cations comprising at least potassium, and anions comprising at least one of chloride, nitrate and sulphate.

In another preferred embodiment of the FOS composition, the anions comprise at least two of chloride, nitrate and sulphate, and, even more preferred, comprise chloride, nitrate and sulphate.

In yet another embodiment, the FOS composition is characterized in that at least 35 or 40 wt. % of the fructooligosaccharides in the composition have a DP of 3 as measured in HPLC.

In another embodiment, the FOS composition has a color of between 1,000-3,000 Icumsa units, and, more preferably a conductivity of between 200-2,000 μS/cm.

The FOS composition may contain minerals, vitamins, amino acids or proteins, if desired. These compounds may be added to the FOS composition, or they may already integrally be contained in the raw materials used to prepare the FOS composition.

The FOS composition as such, or obtainable by the method of the invention, may conveniently be used as food ingredient in foodstuffs, or in pet food or animal feed.

The invention will now be illustrated by means of the following examples, without however being limited thereto.

### EXAMPLE 1

Sucrose supplied by Tiense Suikerraffinaderij from refined sugar origin was combined with water and an enzyme to form an aqueous mixture. After solubilizing, the solids content was 60° Bx. The enzyme used was Novozyme 960, an endo-inulinase enzyme in non-immobilized form. The activity of the Novozyme 960 enzyme was 306 U per gram. One unit U of an endo-inulinase enzyme corresponds to the amount of enzyme that is capable of liberating 1 μmol reducing sugar per minute from an inulin sample. The amount of Novozyme 960 enzyme used in the mixture was 0.425 U per gram dry matter of raw material.

The mixture was brought to a pH of 6.2 by adding H₂SO₄ and/or NaOH to the aqueous mixture. After filtering over a sieve with mesh size 3 mm to remove larger impurities, the mixture was re-solubilised at a temperature of 80° C., lowered to a temperature of between 57 and 61° C. and left to react for about 20 hours. After the said about 20 hours, the reaction was ended by increasing the pH to a value of above 8.5 to stop enzymatic activity. After said reaction, about 58-60 wt % of FOS was formed in the mixture, with the remaining 40-42 wt % being other carbohydrates like sucrose and glucose.

In a subsequent step, the aqueous mixture containing the FOS and other carbohydrates was subjected to a chromatographic separation step in which a portion of the non-FOS carbohydrates was separated from the aqueous mixture using a cationic resin, in particular Applexion XA 2004/32 K, a styrene divinylbenzene copolymer obtained from Novasep. Water was used as eluent.

The chromatographic separation step was performed at a temperature of 70° C. during a residence time of about 6-8 hrs, at a pH within the range 8-9. After chromatographic separating a portion of non-FOS carbohydrate molecules from the aqueous mixture, a FOS-enriched stream and composition was obtained having about 85 wt. % FOS relative to the total amount of carbohydrates in the aqueous mixture. The solid content was about 15° Bx.

In a last step, the FOS-enriched stream of the aqueous mixture was evaporated to yield a syrupy FOS composition of at least 72° Bx in the present example.

The obtained FOS composition was analysed by means of HPLC. The results are summarized in Table 1 below.

TABLE 1

| | | |
|---|---|---|
| °Bx | | 76.0 |
| Fructose | g/ 100 g Bx | 1.7 |
| Glucose | g/ 100 g Bx | 3.7 |
| FOS, DP2 | g/ 100 g Bx | 0.2 |
| Saccharose | g/ 100 g Bx | 6.9 |
| FOS, F3 | g/ 100 g Bx | 0.7 |
| FOS, DP3 | g/ 100 g Bx | 42.9 |
| FOS, DP4 | g/ 100 g Bx | 36.4 |
| FOS, DP5 and higher | g/ 100 g Bx | 7.0 |
| Total Carbohydrates | g/ 100 g Bx | 99.5 |
| Total Inuline / Oligofructose | g/ 100 g Bx | 87.3 |

In Table 1, the amounts of the various compounds are given in weight percentage of total dry carbohydrate matter. Further, the terms DP2, DP3, DP4 and DP5 indicate FOS compounds having a degree of polymerisation of 2, 3, 4 and 5, respectively. It is seen that the FOS composition comprises a major amount of DP3 and DP4 compounds. Such a FOS is also referred to as a short chain FOS or scFOS.

The syrupy FOS composition had an Icumsa color of 2800 Icumsa and a conductivity of 600 μS/cm.

It turned out that the syrupy FOS composition as prepared by the claimed method comprises at least 1 g/kg ° Bx of a mixture of organic acids and ions. Table 2 summarizes this mixture:

TABLE 2

| | | |
|---|---|---|
| Cations: | | |
| Sodium | mg/ kg Bx | 650 |
| Ammonium | mg/ kg Bx | 1 |
| Potassium | mg/ kg Bx | 899 |
| Magnesium | mg/ kg Bx | <1 |
| Calcium | mg/ kg Bx | <1 |
| Anions: | | |
| Chloride | mg/ kg Bx | 47 |
| Nitrite | mg/ kg Bx | <1 |
| Nitrate | mg/ kg Bx | 46 |
| Phosphate | mg/ kg Bx | 7 |
| Sulphite | mg/ kg Bx | <1 |
| Sulphate | mg/ kg Bx | 533 |
| Organic acids: | | |
| PCA | mg/ kg Bx | 210 |
| Lactate | mg/ kg Bx | 296 |
| Acetate | mg/ kg Bx | 725 |
| Propionate | mg/ kg Bx | 5 |
| Formiate | mg/ kg Bx | 181 |
| Butyrate | mg/ kg Bx | 3 |
| Malate | mg/ kg Bx | 4 |
| Oxalate | mg/ kg Bx | 16 |
| Citrate | mg/ kg Bx | 479 |
| Sum cations + anions + organic | mg/ kg Bx | 4101 |
| acids | | |
| Sum cations + anions + organic | g/ kg Bx | 4.1 |
| acids | | |

The syrupy FOS composition of the invention was first stored for 1 year at a temperature ranging between 10-20° C. The stability of the syrupy FOS composition was then determined by subsequently storing it at 25° C. and measuring its pH and color at 25° C. over time, i.e. following the initial storage time of 1 year. The results are shown in Table 3 below:

TABLE 3

| Days | 0 | 27 | 56 | 87 |
|---|---|---|---|---|
| pH | 6.26 | 6.17 | 6.03 | 6.03 |
| Color (Icumsa Units) | 2625 | 2633 | 2561 | 2525 |

The results show a surprisingly constant pH, even after 1 year and 87 days. The color did not change appreciably; after 1 year and 87 days there was hardly any degradation of FOS compounds (less than 0.5 g/100 g Bx). This is all evidence of a highly stable FOS product.

### Comparative Experiment A

An aqueous FOS composition having the same ° Bx and essentially the same profile of FOS compounds as in Example 1—albeit with a slightly lower FOS content—but having a low content of organic acids and ions as is characteristic for known commercial scFOS products, was provided. The sum of cations, anions and organic acids was determined to be 17 mg/kg Bx.

The stability of the FOS composition was determined by storing it at 25° C. and measuring its pH and FOS content at 25° C. over time. The results are shown in Table 4 below:

TABLE 4

| Days | 0 | 62 | 109 | 139 |
|---|---|---|---|---|
| pH | 5.03 | 4.57 | 4.24 | 4.06 |
| Total FOS (g/ 100 g Bx) | 85.4 | 84.7 | 83.3 | 81.2 |

The results show a relatively strong decrease of pH of about 20% after 139 days. The results also show that the FOS in the FOS composition is subject to degradation (presumably hydrolysis) and diminishes from 85,4° Bx to 81,2° Bx after 139 days. This is evidence of a clearly unstable FOS product.

The invention relates in particular to the following series of embodiments i) to xxviii):
i) Method for producing a stable fructooligosaccharide (FOS) composition, comprising the steps of:
   a) providing a raw material containing sucrose;
   b) forming an aqueous mixture of the raw material with an enzyme, whereby the enzyme at least has fructosyltransferase activity;
   c) exposing the formed aqueous mixture to conditions whereby FOS-forming takes place until the amount of non-FOS carbohydrates in the mixture, such as fructose, glucose and sucrose, constitute at most 50 wt. % of the total amount of carbohydrates in the mixture;
   d) optionally deactivating the enzyme;
   e) chromatographically separating non-FOS carbohydrates from the aqueous mixture using a resin to yield a FOS-enriched stream containing at least 75 wt. % FOS relative to the total amount of carbohydrates in the aqueous mixture and a stream enriched in non-FOS carbohydrates, and whereby the FOS-enriched stream comprises at least 100 mg/kg ° Bx of organic acids and ions; and
   f) evaporating the FOS-enriched stream to yield a syrupy FOS composition of at least 65° Bx,
   wherein at least part or essentially all of the organic acids and ions is formed in situ during any one of the steps c) to f).
ii) Method according to embodiment i), wherein the organic acids and ions are formed in situ during step e).
iii) Method according to embodiment ii), wherein in step e) the FOS-enriched stream is brought to a pH within a range of 6.0 to 11.0, preferably 8.0 to 10.0, to form the FOS-enriched stream comprising at least 100 mg/kg ° Bx of organic acids and ions.
iv) Method according to embodiments ii) and iii), wherein a portion of the stream enriched in non-FOS carbohydrates is brought to a pH within a range of 6.0 to 11.0, preferably 8.0 to 10.0, and subsequently recombined with the FOS-enriched stream to form the FOS-enriched stream comprising at least 100 mg/kg ° Bx of organic acids and ions.
v) Method according to embodiments iii) and iv), wherein the pH is held within the range for a residence time of between ten minutes and eight hours.
vi) Method according to any one the preceding embodiments, wherein the pH of the FOS-enriched stream and/or of the syrupy FOS composition is brought to below 8.0, more preferably within a range of 6.0 to 8.0, after step e) or step f).
vii) Method according to any one the preceding embodiments, wherein the amount of the organic acids and ions in the FOS-enriched stream comprises at least 200 mg/kg ° Bx, more preferably at least 300 mg/kg ° Bx, even more preferably at least 400 mg/kg ° Bx, even more preferably at least 500 mg/kg ° Bx, even more preferably at least 1000 mg/kg ° Bx.
viii) Method according to embodiment vii), wherein the FOS-enriched stream comprises between 1-10 g/kg ° Bx, more preferably between 1.2-8 g/kg ° Bx, and most preferably between 1.5-5 g/kg ° Bx of the organic acids and ions.
ix) Method according to any one of embodiments i)-viii), wherein in step e) the resin is a cationic resin.
x) Method according to any one of embodiments i)-ix), wherein the cationic resin comprises at least part of the organic acids and ions.
xi) Method according to any one of embodiments i)-x), wherein at least part of the organic acids and ions are added to the aqueous mixture after step e), during step f) or after step f).
xii) Method according to any one of the preceding embodiments, wherein the organic acids comprise at least one of pyrrolidone carboxylic acid (PCA), lactate, acetate, and formiate.
xiii) Method according to embodiment xii), wherein the organic acids comprise citrate.
xiv) Method according to any one of the preceding embodiments, wherein at least acetate is not added to the aqueous mixture.
xv) Method according to any one of the preceding claims, wherein the ions comprise cations comprising at least one of sodium and potassium, and anions comprising at least one of chloride, nitrate and sulphate.
xvi) Method according to any one of the preceding embodiments, wherein the cationic resin comprises a styrene divinylbenzene copolymer.
xvii) Method according to any one of the preceding embodiments, wherein the conditions whereby FOS-forming can take place include a temperature between 40° C.-75° C. and a solids content lying between 40° Bx and 70° Bx, more preferably 45 and 70° Bx, more preferably 50 and 70° Bx.
xviii) Method according to embodiment xvii), wherein the temperature lies between 50° C.-65° C. and the solids content lies between 50° Bx and 65° Bx.
xix) Method according to any one of the preceding embodiments, wherein the fructooligosaccharide (FOS) composition has a color of between 1,000-3,000 Icumsa units.
xx) Method according to any one of the preceding embodiments, further including a step g) after step e) and before step f) of treating the FOS-enriched stream with active carbon to remove at least part of colored components present in the FOS-enriched stream without substantially affecting the minimum amount of 1 g/kg ° Bx of organic acids and ions in the FOS.
xxi) Method according to embodiment xx), wherein treating the aqueous mixture with active carbon yields a color of between 50-750 Icumsa units, more preferably of between 100-500 Icumsa units, and most preferably of between 150-400 Icumsa units.
xxii) Method according to any one of the preceding embodiments, not including any further steps after step f) or step g).
xxiii) Fructooligosaccharide (FOS) composition wherein the composition comprises at least 75 wt. % FOS relative to the total amount of carbohydrates, and wherein the FOS composition comprises at least 100 mg/kg ° Bx of organic acids and ions.
xxiv) FOS composition according to embodiment xxiv), wherein the organic acids comprise at least one, and preferably at least two, of pyrrolidone carboxylic acid (PCA), acetate, and formiate.
xxv) FOS composition according to embodiment xxiii) or xxiv), not comprising at least one of citrate and acetate.
xxvi) FOS composition according to any one of embodiments xxiii)-xxv), wherein the ions comprise cations comprising at least one of sodium and potassium, and anions comprising at least one of chloride, nitrate and sulphate.
xxvii) FOS composition according to any one of embodiments xxiii)-xxvi), wherein at least 35 wt. % of the fructooligosaccharides in the composition have a DP of 3 as measured in HPLC.
xxviii) FOS composition according to any one of embodiments xxiii)-xxvii), having a color of between 1,000-3,000 Icumsa units.
xxix) FOS composition according to any one of embodiments xxiii)-xxviii), having a conductivity of between 200-2,000 μS/cm.
xxx) FOS composition according to any one of embodiments xxiii)-xxix), which is a scFOS composition.
xxxi) Use of the FOS composition according to any one of embodiments xxiii)-xxx) in foodstuffs, pet food, or in animal feed.
xxxii) FOS composition according to any one of embodiments xxiii)-xxx) or syrupy scFOS composition obtainable according to any one of embodiments i)-xxii), wherein the sum of pyrrolidone carboxylic acid (PCA), lactate, acetate, formiate, and citrate accounts for at least 50 wt. % of all organic acids in the FOS composition or syrupy scFOS composition.

1. A method for producing a stable fructooligosaccharide (FOS) composition, comprising the steps of:
a) providing a raw material containing sucrose;
b) forming an aqueous mixture of the raw material with an enzyme, whereby the enzyme at least has fructosyltransferase activity;
c) exposing the formed aqueous mixture to conditions whereby FOS-forming takes place until the amount of non-FOS carbohydrates in the mixture, constitute at most 50 wt. % of the total amount of carbohydrates in the mixture;
e) chromatographically separating the non-FOS carbohydrates from the aqueous mixture using a resin, whereby organic acids and ions are formed and whereby the resin provides or facilitates the formation of at least part of the organic acids and ions, to yield a FOS-enriched stream containing at least 75 wt. % FOS relative to the total amount of carbohydrates in the aqueous mixture and a stream enriched in non-FOS carbohydrates, and whereby the FOS-enriched stream comprises between 1.2-8 g/kg dry matter of organic acids and ions, the organic acids comprising at least three of pyrrolidone carboxylic acid (PCA), lactate, acetate and formiate; and
f) evaporating the FOS-enriched stream to yield a syrupy FOS composition of at least 65° Bx, wherein at least part or all of the organic acids and ions is formed during step e), and wherein, to form the FOS-enriched stream comprising between 1.2-8 g/kg dry matter of organic acids and ions, the FOS-enriched stream is brought to a pH within a range of 8.0 to 11.0 in step e).

2. The method as claimed in claim 1, wherein the pH is held within the range in step e) for a residence time of between six and eight hours.

3. The method as claimed in claim 1, wherein the pH of the FOS-enriched stream and/or of the syrupy FOS composition is brought to below 8.0, after step e) or step f).

4. The method as claimed in claim 3, wherein the pH is brought to a range of 6.0 to 8.0.

5. The method as claimed in claim 1, wherein the FOS-enriched stream comprises between 1.5-5 g/kg dry matter of the organic acids and ions.

6. The method as claimed in claim 1, wherein in step e) the resin is a cationic resin.

7. The method as claimed claim 1, wherein the ions comprise cations comprising at least one of sodium and potassium, and anions comprising at least one of chloride, nitrate and sulphate.

8. The method as claimed in claim 1, wherein the conditions whereby FOS-forming takes place include a temperature between 50° C.-65° C. and the solids content lies between 50° Bx and 65° Bx.

9. The method as claimed in claim 1, further including a step g) after step e) and before step f) of treating the FOS-enriched stream with active carbon to remove at least part of colored components present in the aqueous mixture without affecting the between 1.2-8 g/kg dry matter of organic acids and ions in the FOS-enriched stream.

10. The method as claimed in claim 1, further comprising, after step c), a step d) of deactivating the enzyme.

11. The method as claimed in claim 2, wherein the pH of the FOS-enriched stream and/or of the syrupy FOS composition is brought to below 8.0, after step e) or step f).

12. The method as claimed in claim 11, wherein the pH is brought to a range of 6.0 to 8.0.
